(19) United States
(12) Patent Application Publication
(10) Pub. No.: US 2020/0123271 A1
(43) Pub. Date: Apr. 23, 2020

(54) COMPOSITION FOR ENHANCING SENSITIVITY OF PROSTATE CANCER TO ANTICANCER DRUG AND USE THEREOF

(71) Applicant: KOREA INSTITUTE OF SCIENCE AND TECHNOLOGY, Seoul (KR)

(72) Inventors: Kwan Hyi LEE, Seoul (KR); Hyojin LEE, Seoul (KR)

(73) Assignee: KOREA INSTITUTE OF SCIENCE AND TECHNOLOGY, Seoul (KR)

(21) Appl. No.: 16/594,350

(22) Filed: Oct. 7, 2019

(30) Foreign Application Priority Data
Oct. 22, 2018 (KR) 10-2018-0126309

(51) Int. Cl. C07K 16/32; C12N 15/113; A61K 31/277; A61K 38/09; A61K 31/565; A61K 31/136; C07K 16/22; C07K 16/28; A61P 35/00; G01N 33/50

(52) U.S. Cl. CPC C07K 16/32; C12N 15/1135; A61K 31/277; A61K 38/09; A61K 31/565; C12N 2310/20; C07K 16/22; C07K 16/2863; A61P 35/00; G01N 33/5011; C07K 2317/76; A61K 31/136

(57) ABSTRACT

Provided is a composition for enhancing the sensitivity of prostate cancer to an anti-cancer drug. The composition may overcome resistance of cancer cells to a target anti-cancer drug and improve not only sensitivity of the cancer cells to the anti-cancer drug but also therapeutic effects on prostate cancer via combined administration with a conventional target anti-cancer drug.

### CROSS-REFERENCE TO RELATED APPLICATION

This application claims the benefit of Korean Patent Application No. 10-2018-0126309, filed on Oct. 22, 2018, in the Korean Intellectual Property Office, the disclosure of which is incorporated herein in its entirety by reference.

### REFERENCE TO SEQUENCE LISTING SUBMITTED VIA EFS-WEB

This application includes an electronically submitted sequence listing in .txt format. The .txt file contains a sequence listing entitled “1183-0137PUS1_ST25.txt” created on Nov. 27, 2019 and is 1,608 bytes in size. The sequence listing contained in this .txt file is part of the specification and is hereby incorporated by reference herein in its entirety.

### BACKGROUND

### 1. Field

One or more embodiments relate to a composition for enhancing sensitivity of prostate cancer to an anti-cancer drug.

### 2. Description of Related Art

Androgen-independent prostate cancer (AIPC) is a subtype of prostate cancer that develops despite androgen-deprivation therapy. In the case where a median survival time of 10 months to 20 months is expected after diagnosis, effective treatment of AIPC is required. Although there are several options for secondary hormone treatment and chemotherapy, effects thereof last for a short period of time and the overall survival rate does not considerably increase. Also, secondary hormone treatment of patients with chemotherapy using aminogluthetimide, high-dose bicalutamide, megestrol acetate, hydrocortisone, prednisone, ketoconazole and mitoxantrone, satraplain, a platinum agent, and the like has not increased the survival rates of patients. Therefore, there is a need to develop a novel anti-cancer drug capable of detecting the emergence of resistance to anti-cancer drugs early in the treatment process and overcome the resistance.

### SUMMARY

One or more embodiments include a composition for enhancing the sensitivity of prostate cancer to an anti-cancer drug, the composition including a substance capable of inhibiting the expression or activity of ERG.

One or more embodiments include an anti-cancer adjuvant for prostate cancer, the anti-cancer adjuvant including a substance capable of inhibiting expression or activity of ERG.

One or more embodiments include a composition for treating prostate cancer resistant to an anti-cancer drug, the composition including a substance capable of inhibiting expression or activity of ERG.

One or more embodiments include a method of screening an anti-cancer drug, the method including: brining a biological sample isolated from a patient with prostate cancer into contact with a test substance; measuring an expression level of ERG gene or an amount of ERG protein in the sample in contact with the test substance; and comparing the measured gene expression level or protein amount with a gene expression level or a protein amount of a control.

One or more embodiments include a method of providing information for predicting a prognosis of prostate cancer, the method including: measuring an expression level of ERG gene or an amount of ERG protein in a biological sample isolated from a patient with prostate cancer; and comparing the measured gene expression level or protein amount with a gene expression level or a protein amount of a control.

One or more embodiments include a method of enhancing sensitivity of prostate cancer to an anti-cancer drug, the method including administering a substance inhibiting expression or activity of ERG to an individual in need thereof.

One or more embodiments include a method of treating prostate cancer, the method including administering an anti-cancer adjuvant for prostate cancer including a substance inhibiting expression or activity of ERG to an individual in need thereof.

One or more embodiments include a method of treating prostate cancer resistant to an anti-cancer drug, the method including administering a substance inhibiting expression or activity of ERG to an individual in need thereof.

Additional aspects will be set forth in part in the description which follows and, in part, will be apparent from the description, or may be learned by practice of the presented embodiments of the disclosure.

According to one or more embodiments, provided is a composition for enhancing sensitivity of prostate cancer to an anti-cancer drug, the composition including a substance inhibiting expression or activity of ERG.

According to one or more embodiments, provided is an anti-cancer adjuvant for prostate cancer, the anti-cancer adjuvant including a substance inhibiting expression or activity of ERG.

According to one or more embodiments, provided is a composition for treating prostate cancer resistant to an anti-cancer drug, the composition including a substance inhibiting expression or activity of ERG.

The prostate cancer may be androgen-independent prostate cancer. The prostate cancer may also be cancer having resistance to anti-cancer drugs or immunoresistance. The composition may enhance sensitivity to an anti-cancer drug, resulting in improvement of therapeutic effects of the anti-cancer drug. In addition, because a dosage amount of the anti-cancer drug used in combination may be reduced by enhancing sensitivity to the anti-cancer drug, side effects of the anti-cancer drug may be reduced.

Throughout the specification, the term “sensitivity to an anti-cancer drug” refers to the degree of response of cancer cells to the anti-cancer drug. Since therapeutic effects of the anti-cancer drug on cancer cells resistant to an anti-cancer drug decrease due to repeated administration thereof, an anti-cancer drug targeting ERG may be used as a novel anti-cancer drug to treat prostate cancer having anti-cancer drug resistance.

As used herein, the term “anti-cancer adjuvant” refers to an agent capable of improving, enhancing, or increasing anti-cancer effects of the anti-cancer drug. In general, although the anti-cancer adjuvant does not have anti-cancer effects by itself, anti-cancer effects of the anti-cancer drug may be improved, enhanced, or increased when the anti-cancer drug is used together with the anti-cancer adjuvant. Meanwhile, the anti-cancer adjuvant according to an embodiment has an effect of enhancing anti-cancer activity when administered in combination with the anti-cancer drug.

The substance may be an antisense oligonucleotide, an aptamer, siRNA, shRNA, or miRNA of ERG. The substance may also be an antibody or an antigen-binding fragment thereof capable of inhibiting the activity of a protein encoded by the ERG. Any antibody specifically binding to the protein encoded by the ERG may also be used. The antibody may be, for example, not only monoclonal antibody, chimeric antibody, humanized antibody, human antibody, but also any functional fragments of the antibody. Also, the antibody may be not only a complete form having a full length of two heavy chains and two light chains, but also a functional fragment of the antibody, as long as the antibody has a binding property of specifically recognizing a protein encoded by ERG. The functional fragment of the antibody molecule refers to a fragment including at least an antigen-binding function and may be Fab, F(ab′), F(ab′)2, Fv, and the like.

The anti-cancer drug may be a taxane-based compound. For example, the anti-cancer drug may be Docetaxel (taxotere) or Paclitaxel (Taxol). Also, the anti-cancer drug may be casodex, zoladex, Lupron, estramustine, Mitoxantrone, or Tannock.

According to an embodiment, the composition may further include a therapeutic agent having therapeutic effects on prostate cancer in addition to the oligonucleotide or the antibody each inhibiting the expression or activity of the protein encoded by ERG, and used as an active ingredient. The composition may further include a pharmaceutically acceptable carrier, excipient, or diluent according to an administration method thereof. Specifically, the composition may further include saline, sterilized water, Ringer's solution, buffered saline, a dextrose solution, a maltodextrin solution, glycerol, ethanol, liposome, and any combination thereof, and may further be supplemented with any other common additive such as an antioxidant and a buffer solution, if required. In combination with a diluent, a dispersant, a surfactant, a binder, a lubricant, or the like, the composition may be formulated into injectable dosage forms, such as aqueous solutions, suspensions, and emulsions, pills, capsules, granules, and tablets. Also, a target organ or tissue-specific antibody or other ligands may be used in combination with the carrier to specifically act on the target organ. These types of carriers, excipients, or additives encompass all formulations commonly available in the art, without being limited thereto.

According to purposes or needs, the composition or mixture may appropriately be administered to an individual according to any method, administration route, and dosage amount well known in the art. Examples of the administration route may include oral, parenteral, subcutaneous, intraperitoneal, intrapulmonary, and intranasal routes. For topical treatment, the composition may be administered by a suitable method including intralesional injection. The parental administration may include intramuscular, intravenous, intraarterial, intraperitoneal, or subcutaneous administration. In addition, an appropriate dose and administration frequency may be selected according to any method well known in the art, and the amount of the composition according to the present disclosure actually administered and including the antisense oligonucleotide, aptamer, siRNA, shRNA, or miRNA may appropriately be determined by various factors such as types of symptoms to be prevented or treated, administration route, gender, health condition, diet, age, and weight of an individual, and severity of disease.

According to an embodiment, the composition may further include a substance inhibiting expression or activity of TMPRSS2-ERG. Details of the substance that inhibits expression and activity are as described above.

According to one or more embodiments, provided is a method of screening an anti-cancer drug, the method including: brining a biological sample isolated from an individual into contact with a test substance; measuring an expression level of ERG gene or an amount of ERG protein in the sample in contact with the test substance; and comparing the measured gene expression level or protein amount with a gene expression level and a protein amount of a control. According to an example embodiment, the method may further include measuring an expression level of TMPRSS2-ERG gene or an amount of TMPRSS2-ERG protein; and comparing the measured gene expression level or protein amount with a gene expression level or a protein amount of a control.

The test substance may be an individual nucleic acid, protein, extract, or the like, as a natural product or a compound, randomly selected or assumed to have a potential as an anti-cancer drug or a therapeutic agent for cancer with immunoresistance according to a conventional screening method. Specifically, a test substance that reduces the expression level of ERG gene or the amount of ERG protein obtained therefrom in the biological sample of the individual in contact with the test substance may be determined as a therapeutic agent for prostate cancer. The anti-cancer drug determined by the method may serve as a leading compound in a subsequent process of developing the therapeutic agent. A novel therapeutic agent for anti-cancer drug-resistant cancer may be developed by modifying and optimizing the structure of the leading compound.

In the comparing of the measured gene expression level or protein amount with the gene expression level or protein amount of the control, a biomarker of anti-cancer drug-resistant prostate cancer may be ERG protein or TMPRSS2-ERG protein. The ERG protein or TMPRSS2-ERG protein may be used as the biomarker by measuring changes in decreased expression levels of ERG protein and TMPRSS2-ERG protein. In this regard, although the changes in the expression level of the biomarker of anti-cancer drug-resistant prostate cancer may be an increase or decrease in the expression level, the expression level of ERG or TMPRSS2-ERG may preferably decrease as a result of treatment in the case of a therapeutic agent. The expression level of the biomarker may decrease by a statistically significant level compared to the control.

According to one or more embodiments, provided is a method of providing information for predicting a prognosis of prostate cancer, the method including: measuring an expression level of ERG gene or an amount of ERG protein in a biological sample isolated from an individual; and comparing the measured gene expression level or protein amount with a gene expression level or a protein amount of a control. According to an embodiment, the method may further include: measuring an expression level or activity level of TMPRSS2-ERG; and comparing the measured expression level or activity level with an expression level or activity level of the gene in the control.

Specifically, as the amount of ERG gene or protein expressed in an individual increases, the reactivity of the individual with the anti-cancer drug decreases and the individual may be determined as being in a risk group with a bad survival prognosis. Determining of the individual as being in the risk group with a bed survival prognosis may be performed to predict a relative degree of the survival prognosis according to the anti-cancer drug. For example, the determining may be performed to predict whether the survival prognosis due to resistance to the anti-cancer drug is worse than that of a group in which the expression level of ERG gene or the amount of ERG protein increases. Bad survival prognosis caused by resistance to the anti-cancer drug may indicate a low reactivity to the anti-cancer drug, resulting in deterioration in the survival prognosis. Bad survival prognosis may mean a low survival rate, a short survival time, or a short progressive-free survival time. The low reactivity to the anti-cancer drug may mean resistance to the anti-cancer drug ora low sensitivity to the anti-cancer drug. For example, the determining is performed to predict whether a case in which the expression level of ERG gene or the amount of ERG protein increases indicates the low reactivity to the anti-cancer drug and the bad survival prognosis, when compared with the case in which the expression level of ERG gene or the amount of ERG protein decreases.

As used herein, the term “individual” refers to a patient with prostate cancer for predicting a prognosis of prostate cancer affected by resistance to an anti-cancer drug. The individual may be a vertebrate, e.g., a mammal, an amphibian, a reptile, and a bird, and for example, a mammal, e.g., a human (Homo sapiens), and a Korean. The patient with prostate cancer may be a patient having resistance to an anti-cancer drug due to repetitive administration of the anti-cancer drug.

As used herein, the term “biological sample” may encompass a variety of samples obtained from an individual, such as tissue, tumor tissue, prostate tumor tissue, cells, whole blood, serum, plasm, saliva, sputum, cerebrospinal fluid, or urine.

Methods of detecting the gene may include reverse transcription-polymerase chain reaction (RT-PCR), competitive RT-PCR, real-time RT-PCR), RNase protection assay (RPA), northern blotting, DNA chip assay, and the like.

Methods of detecting the protein may include Western blotting, enzyme linked immunosorbent assay (ELISA), radioimmunoassay (RIA), radio immunodiffusion, ouchterlony immunodiffusion, rocket immunoelectrophoresis, immunohistostaining, immunoprecipitation assay, complement fixation assay, fluorescence activated cell sorter (FACS), protein chip assay, and the like.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A shows graphs illustrating cell viability of NCI-H660 and LNCaP cell lines after cultivation for 24, 48 and 72 hours, respectively, according to casodex dosage;

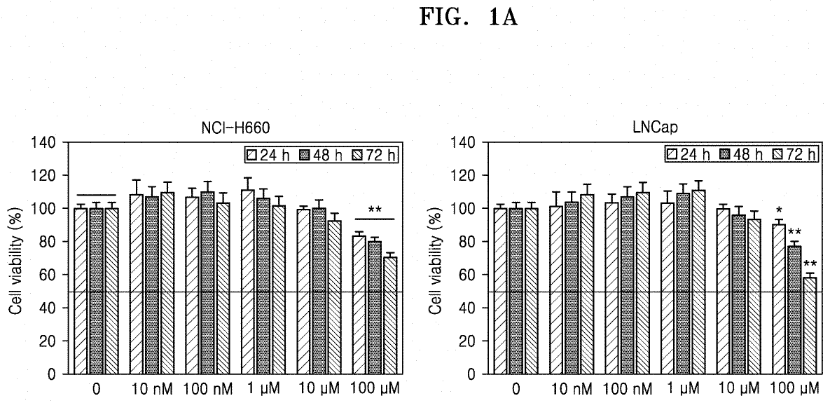

FIG. 1B shows graphs illustrating cell viability of NCI-H660 and LNCaP cell lines after cultivation for 24, 48 and 72 hours, respectively, according to zoladex dosage;

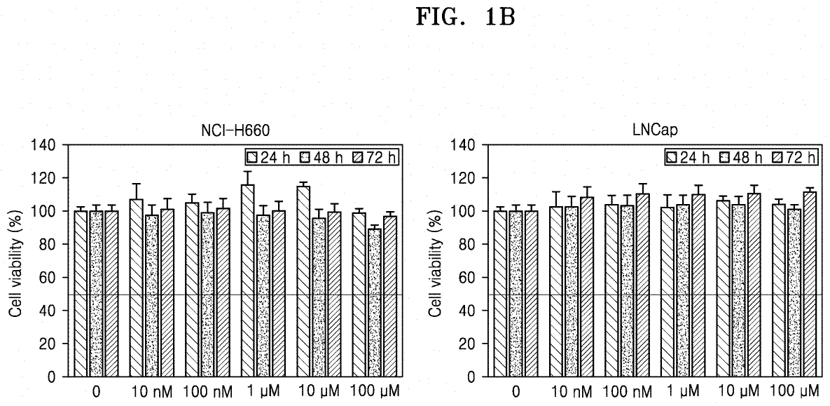

FIG. 1C shows graphs illustrating changes in expression levels of TMPRSS2 and ERG proteins after treating NCI-H660 and LNCaP cell lines with casodex and zoladex, respectively, within 48 hours;

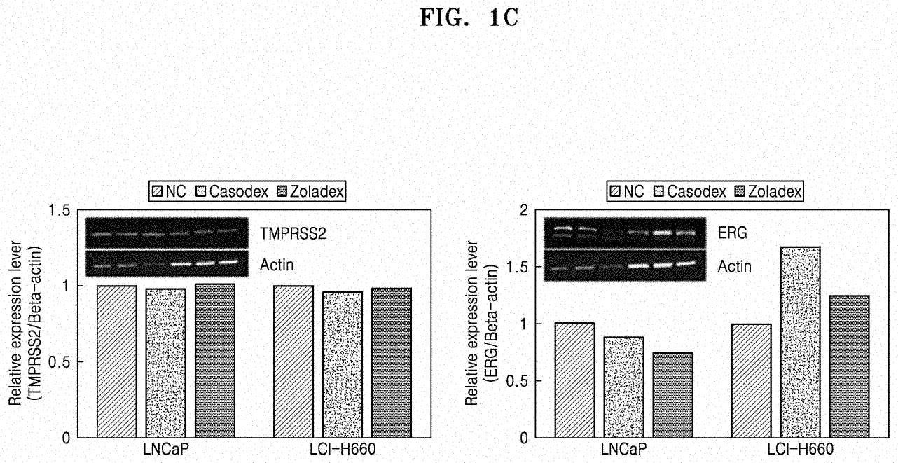

FIG. 1D shows a graph illustrating changes in expression levels of TMPRSS-ERG and protein after treating NCI-H660 and LNCaP cell lines with casodex and zoladex, respectively, for a short period of time;

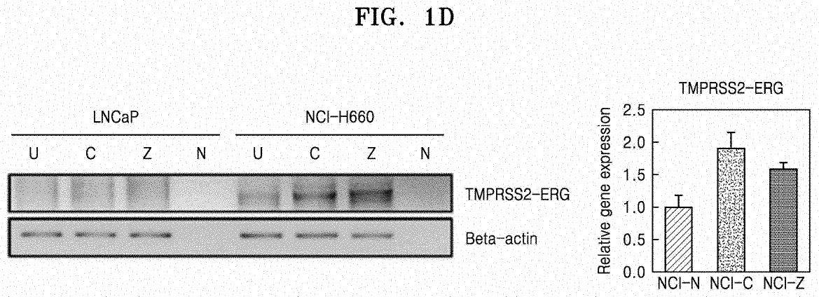

FIG. 2A shows a graph (left) illustrating casodex IC₅₀ measured in an LNCaP cell line and a graph (right) illustrating cell viability of TMPRSS-ERG CRISPR/cas9-mediated knock-out cells in the presence or absence of casodex;

FIG. 2B shows a graph (left) illustrating casodex IC₅₀ measured in an NCI-H660 cell line and a graph (right) illustrating cell viability of ERG or TMPRSS2-ERG CRISPR/cas9-mediated knock-out cells in the presence or absence of casodex;

FIG. 2C shows the expression of the ERG or TMPRSS2-ERG gene in LNCaP and NCI-H660 cell lines not treated, treated with preparation/cas9, and treated with preparation/Cas9/CRISPRsgRNA.

FIG. 2D shows graphs illustrating effects of CRISPR for TMPRSS2-ERG or TMPRSS2-ERG and 30 μM casodex on caspase3/7 expression;

FIG. 3A shows graphs illustrating cell viability and caspase 3/7 expression levels of NCI-H660 cell lines which are not resistant and which are resistant to casodex after single treatment or combined treatment with 30 μM casodex, CRISPRERG, and CRISPRTMPRSS2-ERG.

FIG. 3B shows graphs illustrating cell viability and caspase 3/7 expression levels of LNCaP cell lines which are not resistant and which are resistant to casodex after single treatment or combined treatment with 30 μM casodex, CRISPRERG, and CRISPRTMPRSS2-ERG.

FIG. 3C is a graph illustrating ATP levels in LNCaP cell lines which are not resistant and which are resistant to casodex after single treatment or combined treatment with a pgp inhibitor, casodex, and CRISPR-sgRNA; and

FIG. 4 illustrates the expression of water-soluble alpha tubulin, polymeric alpha tubulin, and beta 3 tubulin genes in LNCaP cell lines not treated, treated with preparation/cas9, and treated with preparation/cas9/ CRISPR-sgRNA.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### EXAMPLES

### Example 1

### Identification of Reactivity of Prostate Cancer Cell Line to Anti-Cancer Drug and Expression of ERG

NCI-H660 and LNCaP cells were seeded on transparent 96-well plates at a density of 10⁴ cells/well. After 24 hours, the cells were treated with 30 μM casodex and 30 μM zoladex, respectively, and cultured for 24 hours, 48 hours, and 72 hours. 10 μL of a CellTiter 96 aqueous one solution cell proliferation assay buffer (Promega, USA) was added thereto, followed by incubation at 37° C. for 1 hour. Then, cell viability was evaluated by measuring absorbance at 485 mm.

### Example 2

### Preparation of TMPRSS-ERG Knockout Cell Line Using

### CRISPR/Cas9

ERG or TMPRSS2-ERG knockout cell line was prepared using CRISPR/Cas9 by using Lipofectamine crisprmax Reagent cas9 nuclease transfection protocol. First, NCI-H660 and LNCaP cells were seeded on transparent 96-well or 6-well plates at densities of 10⁴ cells/well and 2×10⁵ cells/well, respectively. To prepare CRISPR/Cas9 preparation, 5 μL and 125 μL of Opti-MEM medium per each well were multiplied by the total number of wells and added to 15 mL Tube 1. Subsequently, 85 ng and 2500 ng of Cas9 nuclease and 21 ng and 625 ng of sgRNA were respectively added to each well, and finally 0.17 μL and 5 μL of Lipfectamine Cas9 Plus reagent were respectively added to each well and slowly mixed. 5 μL and 125 μL of Opti-MEM were respectively added to new 15 mL-Tube 2, and 0.3 μL and 7.5 μL of the Lipofectamine crisprmax reagent were respectively added to each well and slowly mixed. Subsequently, Tube 1 was slowly mixed with Tube 2 by incubating for 5 minutes. After 10 minute incubation, 10 μL and 250 μL of the preparation were added to each well, followed by cultivation for 48 hours. Nucleotide sequences of CRISPR sgRNA are as shown in Table 1 below.

TABLE 1

| SEQ | | |
|---|---|---|
| ID | CRISPR | |
| NO: | sgRNA | Nucleotide sequence |
| 1 | ERG | 5′TGCCTACGGAACGCCACACCTGG 3′ |
| 2 | TMPRSS2-ERG | 5′AAGGCTTCCTGCCGCGCTCCAGG 3′ |

### Example 3

### Identification of Expression of ERG

To identify whether the ERG gene of the cell line prepared in Example 2 was knocked out, expression of the gene was identified by extracting RNA and synthesizing cDNA.

3-1. Extraction of RNA

NCI-H660 and LNCaP cell lines were seeded on 6-well plates at a density of 2×10⁵ cells/well. After 24 hours, the cells were treated with CRISPR ERG, CRISPR TMPRSS2-ERG, and 30 μM casodex, respectively, for 48 hours. 1 mL of Trizol was added thereto to lyse the cells and the samples were homogenized by voltexing. 100 μL of chloroform was added thereto to separate RNA included in a transparent upper aqueous layer from proteins included in a lower organic layer. The solution was vigorously shaken and incubated at room temperature for 3 minutes. The resultant was centrifuged at 4° C. for 15 minutes at 13,000 rpm to obtain a clean aqueous layer including total RNA. Subsequently, the supernatant of the sample was transferred to a new tube and 2.5 ml of isopropanol was added to each sample to precipitate RNA. The sample was incubated at room temperature for 10 minutes, and then centrifuged at 13,000 rpm at 4° C. for 15 minutes to obtain pellets of total RNA. Then, the supernatant was decanted and RNA was washed with 1 mL of 75% ethanol. The sample was centrifuged at 13,000 rpm at 4° C. for 10 minutes again, the supernatant was decanted, and RNA pellets were dried for 10 minutes. RNA was resuspended in RNase-free water and absorbance of RNA was measured at 260 nm to determine a concentration of RNA.

3-2. PCR

cDNA of each sample was synthesized using a cDNA synthesis kit (Takara, Japan). PCR was performed using a PCR master mix (Promega, USA). Primers used in the PCR are shown in Table 2 below.

TABLE 2

| SEQ | | | |
|---|---|---|---|
| ID | | | |
| NO: | Gene | | Primer |
| 3 | ERG | forward | 5′-CCAACAGAGGGCATTTGGCA-3′ |
| | | direction | |
| 4 | | reverse | 5′-GAGCCCCAGTTTTGCTTCCA-3′ |
| | | direction | |
| 5 | TMPRSS2- | forward | 5′-CCTGTGTGCCAAGACGACTG-3′ |
| | ERG | direction | |
| 6 | | reverse | 5′-TTATAGCCCATGTCCCTGCAG-3′ |
| | | direction | |

After performing PCR, a 10× loading buffer (Takara, Japan) was added to PCR products. The PCR products were run in a 2% agarose gel including a 0.01% Diamond Nucleic Acid Dye (Promega, USA) at 100 V for 45 minutes. Nucleic acid was visualized using a gel documentation system.

### Example 4

### Identification of Inhibited Expression of ERG in Anti-Cancer Drug-Resistant Cell Line and Suppressed Growth of Cancer Cells by Combined Treatment with Anti-Cancer Druci

NCI-H660 and LNCaP cells were seeded on transparent 96-well plates at a density of 10⁴ cells/well. After 24 hours, the cells were treated with CRISPR ERG, CRISPR TMPRSS2-ERG, and 30 μM casodex, respectively, for 48 hours. 10 μL of a CellTiter 96 aqueous one solution cell proliferation assay buffer (Promega, USA) was added thereto, followed by incubation at 37° C. for 1 hour. Then, cell viability was evaluated by measuring absorbance at 485 mm.

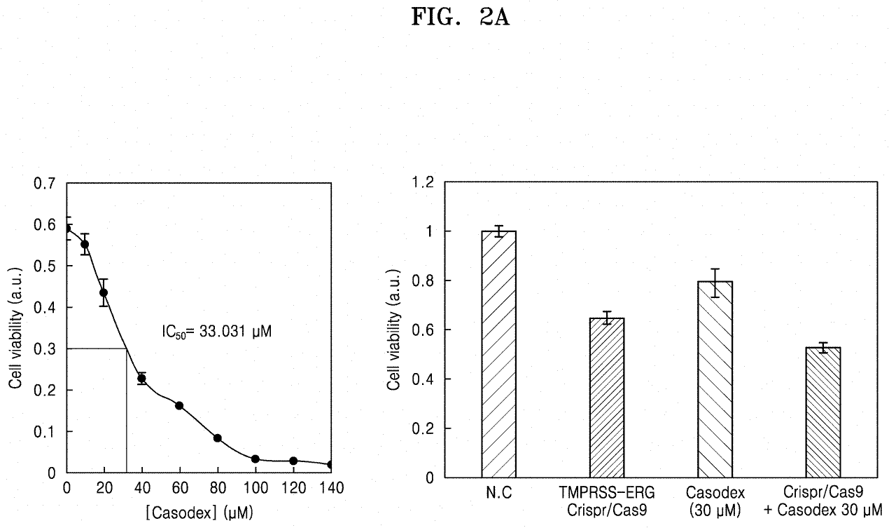

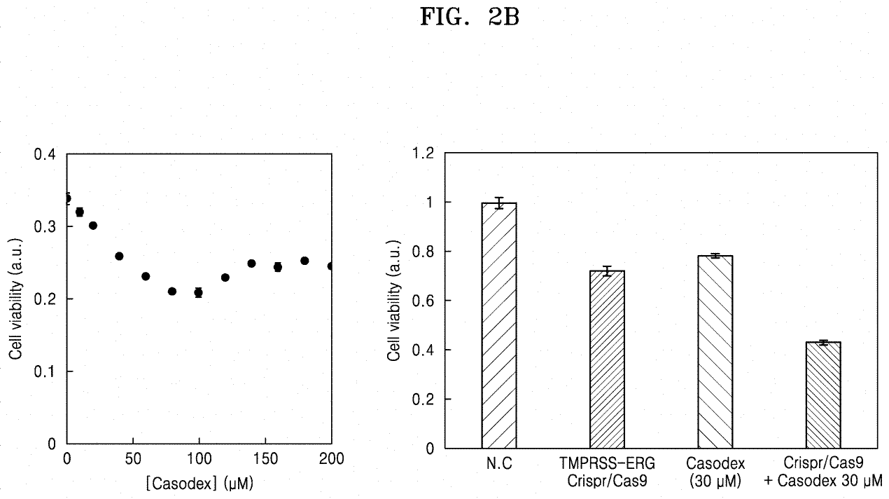

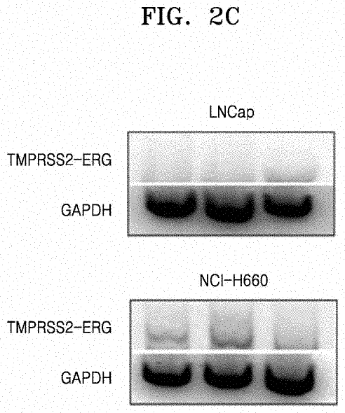

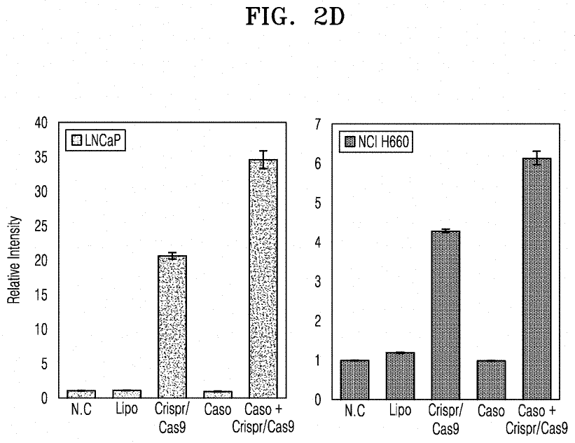

As a result, as shown in FIGS. 2A and 2B, it was confirmed that cell viability was significantly reduced in the cell line treated in combination with casodex when compared with the cell line treated with CRISPR TMPRSS2-ERG. In addition, it was confirmed that cell viability was significantly reduced when the concentration of casodex was in the range of 100 μM to 200 μM.

In addition, as shown in FIG. 2C, it was confirmed that the expression level of TMPRSS2-ERG gene was significantly reduced in the NCI-H660 and LNCaP cell lines treated in combination with casodex.

### Example 5

### Identification of Mechanism According to Inhibition of ERG Expression in Anti-Cancer Drug-resistant Cell Line

5-1. Expression of Caspase 3/7

NCI-H660 and LNCaP cells were seeded on black transparent bottom 96-well plates at a density of 10⁴ cells/well. After 24 hours, the cells were treated with CRISPR ERG, crisprTMPRSS2-ERG, and 30 μM casodex, respectively, for 48 hours. 50 μL of a caspase 3/7 assay buffer (Promega, USA) was added thereto, followed by incubation at room temperature for 1 hour. Then, the activity of caspase 3/7 was detected by measuring luminescence.

As a result, as shown in FIG. 2D, it was confirmed that the expression of caspase 3/7 was significantly increased in the cell line treated with the CRISPR TMPRSS2-ERG. Particularly, it was confirmed that the expression of caspase 3/7 was more significantly increased in the cell line treated in combination with casodex, when compared with the cell line treated with the CRISPR TMPRSS2-ERG.

5-2. Expression of Pgp

NCI-H660 and LNCaP cells were seeded on white transparent bottom 96-well plates at a density of 10⁴ cells/well. After 24 hours, the cells were treated with CRISPR ERG, crisprTMPRSS2-ERG, and 30 μM casodex, respectively, for 48 hours. 20 μL of a pgp-glo assay buffer (Promega, USA) was added to all wells except for Na₃VO₄ condition. 20 μL of 0.25 mM Na₃VO₄ was added to the Na₃VO₄ condition. Reaction was initiated by adding 10 μL of 25 mM MgATP. The plates were mixed for a while and then incubated at 37° C. for 40 minutes. The 40 μL of an ATP detection reagent was added to all wells. Then, the plates were incubated at room temperature for 20 minutes and luminescence was measured.

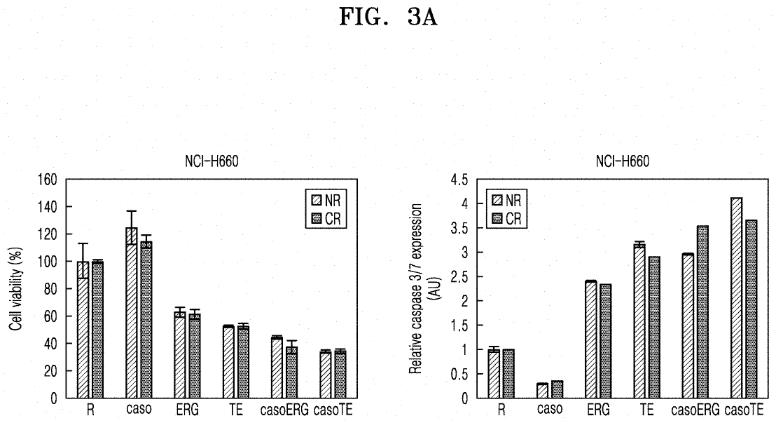

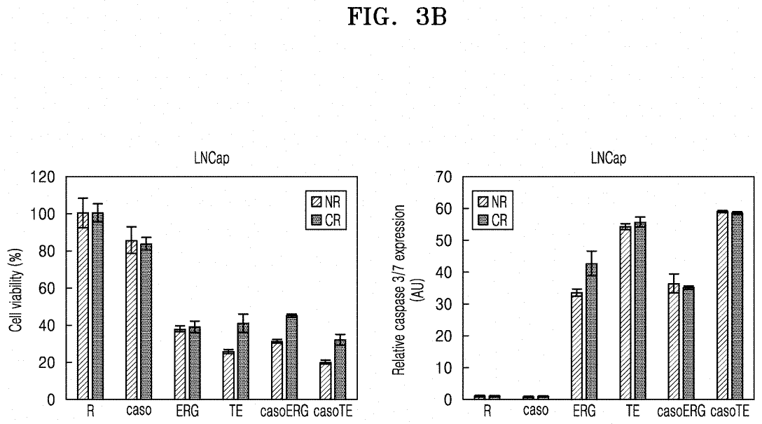

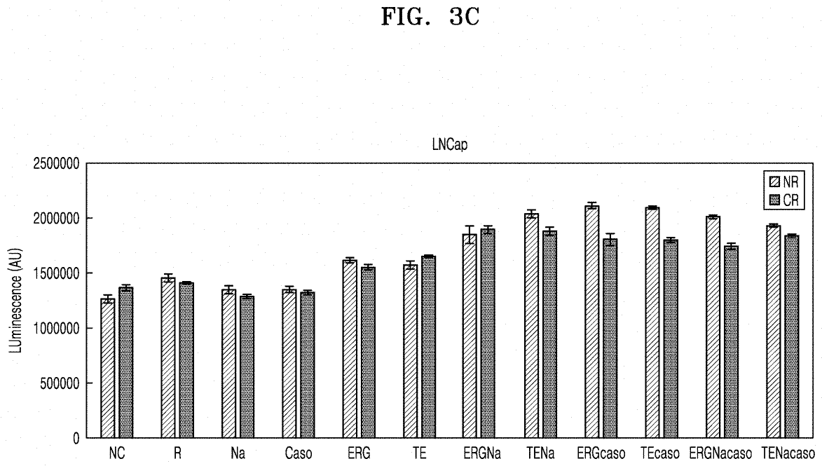

As a result, as shown in FIG. 3C, it was confirmed that ATP was significantly increased in normal and resistant LNCaP cells when ERG or TMPRSS2-ERG was knocked out. Also, it was confirmed that the ATP level was significantly increased in cells when both of ERG and TMPRSS2-ERG were treated in combination with casodex. However, when a pgp inhibitor was additionally used, it was confirmed that the APT level was no longer increased. That is, pgp may be sufficiently suppressed by treating ERG or TMPRSS2-ERG with casodex. In addition, when the normal Lncap cells were compared with resistant LNCaP cells, the ATP level of the resistant cells was lower than that of the normal cells, and thus it was confirmed that pgp was less suppressed in the resistant cells than in the normal cells.

5-3. Activity of Tubulin Protein

NCI-H660 and LNCaP cell lines were seeded on 6-well plates at a density of 2×10⁵ cells/well. After 24 hours, the cells were treated with CRISPR ERG, CRISPR TMPRSS2-ERG, and 30 μM casodex, respectively, for 48 hours. The cells were lysed using Pro-prep protein extraction solution (Intron Biotechnology, South Korea). Cell lysates were quantified using a BCA protein assay kit (Thermo Fisher Scientific, USA).

Then, the expression of proteins was identified by Western blotting. Specifically, SDS 5× loading buffer was added to protein samples diluted to 10-20 μg and heated for 5 minutes. The samples were placed in 4-20% Mini-protean TGX gel (Biorad, USA) and run at 120 V for 60 minutes. Then, the gel was transferred to a nitrocellulose membrane at 300 mA for 90 minutes. The membrane was blocked by 3% BSA in PBS and incubated overnight with a primary antibody in PBS containing 1% BSA at 4° C. The membrane was washed three times with PBS containing with 0.01% Tween 20. A secondary antibody was cultured in a PBS containing 1% BSA for 1 hour at room temperature. The membrane was washed three times with PBS containing 0.01% Tween 20. Then, the membrane was visualized with LAS-3000 (Fujifilm, Japan).

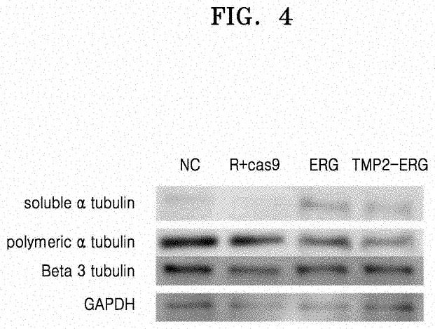

As a result, as shown in FIG. 4, when ERG or TMPRSS2-ERG was knocked out in LNCaP cells, it was confirmed that the amount of water-soluble alpha tubulin increased and the amount of polymeric alpha tubulin decreased. Meanwhile, it was confirmed that there was no significant difference in the case of beta3 tubulin. That is, it may be inferred that ERG and TMPRSS2-ERG may affect formation and maintenance of polymeric alpha tubulin.

The composition according to an embodiment may overcome the resistance of cancer cells to the target anti-cancer drug and improve not only sensitivity of the anti-cancer drug to the cancer cells to the anti-cancer drug but also therapeutic effects for prostate cancer via combined administration with a conventional target anti-cancer drug.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of enhancing the sensitivity of prostate cancer to an anti-cancer drug, the method comprising:
administering a substance which inhibits the expression or activity of ERG to an individual in need thereof.

2. The method of claim 1, wherein
the substance is an antisense oligonucleotide, an aptamer, siRNA, shRNA, or miRNA of ERG.

3. The method of claim 1, wherein
the substance is an antibody or antigen-binding fragment thereof which inhibits the activity of ERG.

4. The method of claim 1, wherein
the prostate cancer is androgen-independent prostate cancer.

5. The method of claim 1, wherein
the anti-cancer drug is a taxane-based compound.

6. The method of claim 1, wherein
the anti-cancer drug is casodex, zoladex, Lupron, estramustine, Mitoxantrone, Avastin, Erbitux, or Tannock.

7. The method of claim 1, further comprising administering a substance
which inhibits the expression or activity of TMPRSS2-ERG.

8. A method of treating prostate cancer, the method comprising
administering an anti-cancer adjuvant comprising a substance which inhibits the expression or activity of ERG to an individual in need thereof.

9. The method of claim 8, wherein
the anti-cancer adjuvant further comprises a substance which inhibits the expression or activity of TMPRSS2-ERG.

10. A method of screening an anti-cancer adjuvant, the method comprising:
bringing a biological sample isolated from a patient with prostate cancer into contact with a test substance;
measuring an expression level of an ERG gene or an amount of ERG protein in the sample in contact with the test substance; and
comparing the measured gene expression level or protein amount with a gene expression level or a protein amount of a control.

11. The method of claim 10, further comprising:
measuring an expression level or activity level of TMPRSS2-ERG;
comparing the measured expression level or activity level of TMPRSS2-ERG with an expression level or activity level of a gene in a control.

12. The method of claim 10, further comprising determining the test substance as a therapeutic agent for prostate cancer when the expression level of the ERG gene or the amount of ERG protein is reduced in the biological sample of the individual in contact with the test substance.


SEQUENCE LISTING (6 sequences)

SEQ ID NO: 1 - DNA, 23 bp, Artificial Sequence
  Synthetic  ERG
tgcctacggaacgccacacctgg

SEQ ID NO: 2 - DNA, 23 bp, Artificial Sequence
  Synthetic  TMPRSS2-ERG
aaggcttcctgccgcgctccagg

SEQ ID NO: 3 - DNA, 20 bp, Artificial Sequence
  Synthetic  Forward primer of ERG
ccaacagagggcatttggca

SEQ ID NO: 4 - DNA, 20 bp, Artificial Sequence
  Synthetic  Reverse primer of ERG
gagccccagttttgcttcca

SEQ ID NO: 5 - DNA, 19 bp, Artificial Sequence
  Synthetic  Forward primer of TMPRSS2-ERG
cctgtgtgccaagacgact

SEQ ID NO: 6 - DNA, 21 bp, Artificial Sequence
  Synthetic  Reverse primer of TMPRSS2-ERG
ttatagcccatgtccctgcag